(19) United States
(12) Patent Application Publication
(10) Pub. No.: US 2003/0134422 A1
(43) Pub. Date: Jul. 17, 2003

(54) Stem cell maturation for all tissue lines

(76) Inventor: Chauncey Bigelow Sayre, Irvine, CA (US)

Correspondence Address: OPPENHEIMER WOLFF & DONNELLY LLP, 840 NEWPORT CENTER DRIVE, SUITE 700, NEWPORT BEACH, CA, 92660, US

(21) Appl. No.: 10/346,816

(22) Filed: Jan. 16, 2003

Related U.S. Application Data
(60) Provisional application No. 60/348,521, filed on Jan. 16, 2002.
(60) Provisional application No. 60/367,161, filed on Mar. 26, 2002.

(51) Int. Cl. C12M 1/00; C12N 15/85; C12N 5/08

(52) U.S. Cl. 435/455; 435/325; 435/366; 435/289.1

(57) ABSTRACT

The present invention provides a composition and method for making a modified germ cell (MGC) comprising a primordial sex cell, (PSC) or nucleus thereof, combined with an enucleated ovum. The PSC is a spermatogonia or an oogonia from a donor animal or mammal. Similarly, the enucleated ovum is from the same species donor animal or mammal as the donor of the PSC. The present invention also provides a method of maturing the MGC in vitro using a specialized cell culture apparatus or bioreactor chamber. The present invention also provides for a method of screening the MGCs for receptor sites, to determine if they are sufficiently matured to be translocated into a host animal or mammal in vivo and/or tissue in vitro. The present invention also provides for a specialized cell culture chamber or bioreactor chamber to grow, expand, maintain, sustain and mature the MGCs, or other types of cells.

### RELATED APPLICATIONS

This application is related to U.S. Provisional Application No. 60/348,521, filed Jan. 16, 2002 and U.S. Provisional Application No. 60/367,161, filed Mar. 26, 2002.

### BACKGROUND OF THE INVENTION

Since the first description of the isolation of embryonic stem (ES) cells from human blastocysts, many reports have surfaced regarding the isolation and characterization of both embryonic stem cells and adult stem cells. Bongso, A. et al. (1996), “Isolation and culture of inner cell mass cells from human blastocyst,” Hum. Reprod. U.S.A. 9, 2110-17.

Stem cells (embryonic and adult) are capable of long-term self renewal and can give rise to mature cell types with specific morphology and function. Similarly, like embryonic stem (ES) cells, which originate from the inner mass of the blastocyst, the origin of adult stem cells share a common origin.

Typically adult stem cells share at least two characteristics: i) they can make identical copies of themselves for long periods of time (long term self-renewal); and they can give rise to mature cell types that have characteristic morphologies and specialized functions. Stem Cells: Scientific Progress and Future Research Directions, Dept. of Health and Human Services, Jun 2001; http://www.nih.gov/news/stemcell/scireport.htm. Adult stem cells are believed to be not as pluripotent as ES cells, however, at least one report has suggested that adult stem cells show more plasticity than previously conceived. Lagasse, E. et al. (2000), “Purified hematopoietic stem cells can differentiate into hepatocytes in vivo,” Nat. Med. 6, 1229-34.

Ultimately, to demonstrate plasticity, an adult stem cell should give rise to fully differentiated cells that have mature phenotypes. The adult stem cells should also be fully integrated into their new tissue environment and be capable of specialized tissue functions, which are appropriate for that tissue. Stem Cells: Scientific Progress and Future Research Directions, supra.

The difficulty in studying adult stem cell plasticity is establishing that the adult stem cell arises out of one type of cell, or cell population. To date, the best studied adult stem cells are based on bone marrow and brain cells. However, studies using stem cells derived from the bone marrow (i.e. hematopoietic stem cells (HSCs), stromal cells and/or endothelial cells) and the brain (i.e. neuroblasts) have their limitations. For example, HSCs from the bone marrow are sorted using a cell sorter, which sorts the cells according to various cell surface markers. This methodology yields highly purified to partially purified cell types. In another example, purification of neuronal stem cells are difficult because these cells are located in different locations (i.e. olfactory bulb, hipppocampus and lateral ventricles of mice) and not in one convenient location or organ tissue. Altman, J. and Das, G. D. (1965), “Autoradiographic and histological evidence of postnatal hippocampal neurogenesis in rats,” J. Compl Neurol., 124, 319-335; Altman, J. (1969), Autoradiographic and histological studies of postnatal neurogenesis. IV. “Cell proliferation and migration into the anterior forebrain, with special reference to persisting neurogenesis in the olfactory bulb,” J. Compl Neurol., 137, 433-457.

Other candidates of adult stem cells are endothelial progenitor cells, skeletal muscle stem cells, epithelial cell precursors in the skin and digestive system and stem cells in the pancreas and liver. Stem Cells: Scientific Progress and Future Research Directions, supra.

Another type of adult stem cell is derived from germ cells, or primordial sex cells (PSC), residing in the lining of the seminiferous tubules of the testes and lining of the ovaries—the spermatogonia and oogonia, respectively. Spermatogonia produce precursor cells that are involved in meiosis. There are at least two types of spermatogonia, type A and type B, and each is easily distinguishable, morphologically and histologically, from the other. For example, type A spermatogonia are more spherical with a prominent nucleolus and uniformly scattered euchromatin. Whereas, type B spermatogonia tend to be more irregular in shape and smaller with a lobed nucleus. Guillaume E, et al. (2001), “Proteome analysis of rat spermatogonia: reinvestigation of stathmin spatio-temporal expression within the testis,” Mol. Reprod. Dev., 60(4):439-45. Chiarini-Garcia, H. and Russell, L. D. (2002), “Characterization of mouse spermatogonia by transmission electron microscopy,” Reproduction, 123(4): 567-77. Thus, unlike other adult stem cells, adult germ cells are easy to locate and distinguished from other interstitial cells.

Similar to other adult somatic stem cells, adult germ cells are diploidy (2n). In contrast to other adult somatic stem cells, germ cells (spermatogonia and oogonia) contain a genome that is undamaged and unspoiled. Whereas, somatic cellular DNA is more damaged (i.e. free radicals) due to their age and low rate of replenishment. Further, somatic stem cells finally succumb to the forces of differentiation that create the tissues of the body. Thus, methods comprising a stem cell consisting of undamaged DNA is preferred.

A persistent problem with adult stem cell transplants in vivo is that of immune rejection. Thus, to date, recipient's of stem cells are reliant on the right donors whose cells will not be rejected by the recipient's immune system. For example, typically bone marrow transplants are allogeneic transplants (different host and donor) and in order for them to work, the recipient's immune system must accept rather than try to destroy the donated marrow. This is accomplished by making sure that the antigens on the donated marrow cells are identical, or very similar to, the antigens on the cells of the recipient. Thus, an improved stem cell transplant method which eliminates concerns regarding immune rejection is highly advantageous.

Therefore, improved methods to provide an adult stem cell which has a high rate of long term self-renewal, while being easy to isolate and purify, and at the same time reduce the associated immune rejection when translocated in vivo or in vitro, will ameliorate existing problems associated with stem cell biology and their use as therapeutics.

### INVENTION SUMMARY

A general object of the present invention is to provide a modified germ cell comprising a primordial sex cell (PSC), or nucleus thereof, translocated into an enucleated ovum, wherein the PSC and the ovum are derived from the same species of animal.

Another general object of the present invention is to provide a method for preparing a modified germ cell comprising: (a) obtaining a PSC from a first donor animal; (b) obtaining an ovum from a second donor animal of the same species as the first donor animal; (c) enucleating the ovum; and (d) translocating the PSC, or nucleus thereof, into the enucleated ovum.

Another general object of the present invention is to provide for maturing a modified germ cell comprising: (a) expanding the modified germ cell in a first medium; (b) placing the modified germ cell into a second medium; (c) screening for membrane receptors on the modified germ cell; (d) repeating steps b and c until the number of receptors on the modified germ cell are sufficient to be a primed modified germ cell; and (f) translocating the primed modified germ cell into the tissue of the animal

Another general object of the present invention is to provide a method for preparing a mammalian modified germ cell comprising: (a) obtaining a PSC from a first donor mammal; (b) obtaining an ovum from a second donor animal of the same species as the first donor animal; (c) enucleating the ovum; (d) translocating the PSC, or nucleus thereof, into the enucleated ovum; (e) expanding the modified germ cell in a first medium; (f) placing the modified germ cell into a second medium; (g) screening for membrane receptors on the modified germ cells; (h) repeating steps b and c until the number of receptors on the modified germ cell are sufficient to be a primed modified germ cell; and (i) translocating the primed modified germ cell into the tissue of the animal.

Another general object of the present invention is to provide a method for preparing a modified germ cell comprising: (a) obtaining a PSC from a first donor animal; (b) obtaining an ovum from a second donor animal of the same species of the first donor animal; (c) enucleating the ovum; (d) fusing the PSC and the enucleated ovum to form a fused cell; and (e) activating the fused cell.

Another general object of the present invention is to provide a method for inducing the modified germ cell to produce the precursor cell that functions in the adult tissue environment, comprising: (a) obtaining at least one modified germ cell; and (b) culturing the modified germ cells in the presence of maturing factors under suitable conditions, and for a time sufficient, to induce the modified germ cells to produce receptor sites.

Another general object of the present invention is to provide a cell culture chamber comprising: (a) at least one isolation chamber; (b) tubing connecting the chamber; and (c) at least one pump.

Still another general object of the present invention is to provide a cell culture chamber comprising: (a) at least one isolation chamber; (b) tubing connecting the chamber; (c) a carbon dioxide source; (d) an oxygen source; (e) a molecular filter; and at least one pump.

### BRIEF DESCRIPTION OF THE DRAWINGS

Detailed description of the preferred embodiment of the invention will be made with reference to the following drawings:

FIG. 1 is schematic drawing of the cell culture or bioreactor chamber.

### DETAILED DESCRIPTION OF THE PREFERRED EMBODIMENTS

This description is not to be taken in a limiting sense, but is made merely for the purpose of illustrating the general principles of the invention. The section titles and overall organization of the present detailed description are for the purpose of convenience only and are not intended to limit the present invention.

The term “primordial sex cell” used herein means a diploid germ cell and/or a spermatogonia and a oogonia.

The term “spermatogonia” used herein means a primordial male sex cells that give rise to progenitors of primary spermatocytes.

The term “oogonia” used herein means a primordial female sex cells that serves as a source of ova.

The term “ovum” used herein means the female gamete, a haploid unfertilized egg, which is capable of developing into a new animal when fertilized by a spermatozoon.

The term “oocyte” used herein means a developing egg cell in oogenesis and upon undergoing meiosis forms the ovum.

The term “bioreactor” used herein means a specialized chamber to grow, expand, maintain, sustain and mature cells in vitro.

The term “modified germ cell” (MGC) used herein means a cell comprised of an ovum cytosol from one animal and a nucleus from either an oogonium or a spermatogonium of the same species animal as that of the ovum, or a different animal.

The term “primed MGC” used herein means modified germ cell(s) that ready for translocation into the host animal in vivo or host tissue in vitro.

The invention provides a totiipotential modified germ cell derived from an animal or mammal donor, which can be expanded, grown, maintained, sustained and matured in a specialized bioreactor chamber. Only those modified germ cells which have the sufficient number of membrane receptors described by the methods herein are within the scope of the invention. Given the methods described herein, the modified germ cells can be made from any animal and translocated into any animal. However, mammals are preferred because there are many therapeutics and beneficial effects of stem cells for mammals. Whereas, the herein described methods are provided for totipotent stem cells, it is possible that similar methods for the expansion, growing, maintaining, sustaining and maturing of embryonic stem cells can be established. Thus, embryonic stem cells and other types of adult stem cells are contemplated in the present invention in the methods described herein.

The present invention also provides for a composition comprising: a mammalian primordial sex cell (PSC), or nucleus thereof, translocated into an enucleated ovum, wherein the PSC and the ovum are derived from the same species of animal or mammal or different animal or mammal. Another term for this composition is a modified germ cell or MGC. In one embodiment, the PSC is a mammalian spermatogonium, or nucleus thereof. In another aspect, the PSC is a mammalian oogonium, or nucleus thereof. Alternative methods of enucleation and nucleation are contemplated within the scope of the present invention including mechanical methods as well as methods utilizing electrical stimuli. The nucleus from any precursor cell from the spermatogonia or oogonia prior to the DNA being divided into the haploid state can be used.

The MGC of the present invention is totipotent, pluripotent, multipotent or bipotent. That is the MGC is capable of forming at least one type of tissue, more particularly, the MGC is capable of forming at least more than one type of tissue.

Once the MGC is established, it can be manipulated by various methods described herein to produce desired characteristics. For example, the MGC can be expanded and maintained in a particular medium. For another example, the MGC can be matured in a step-wise manner to particular stages of development typical of a mature stem cell.

In the step-wise method described herein, the MGC is first expanded to about a 6-cell stage in one chamber of the multi-chamber bioreactor. The MGC can be expanded to more than a 6-cell stage, however, beyond the 10-cell stage, germ cells make progenitor or precursor cells. The 6-cell stage MGC is then matured in a stepwise fashion using cells isolated from different gestation to post-natal stages, which are being maintained in a nearby chamber. At least one group of cells from a gestational to post-natal donor is used to facilitate maturing of the MGC. However, more than one group of cell(s) from a gestational to post-natal stage may be used to mature the MGC. The mature MGC is now termed a primed MGC. The primed MGC has sufficient receptors that upon translocation into a host animal or tissue, in vivo or in vitro, the primed MGC behaves similar to that of a mature stem cell.

Also, provided is a method for screening MGCs which have acquired certain receptors. This screening or quantifying method is described herein using a resonance energy transfer method, in particular, Fluorescence Resonance Energy Transfer (FRET) or Bioluminescence Resonance Energy Transfer (BRET; Packard BioScience, BioSignal Packard Inc., Meriden, Conn.). This method helps to determine which MGCs are ready to be translocated into the host animal.

Other methods to screen for the number of receptors are possible and although not described herein, are within the scope of this invention in that it is used to determine whether the MGC is primed.

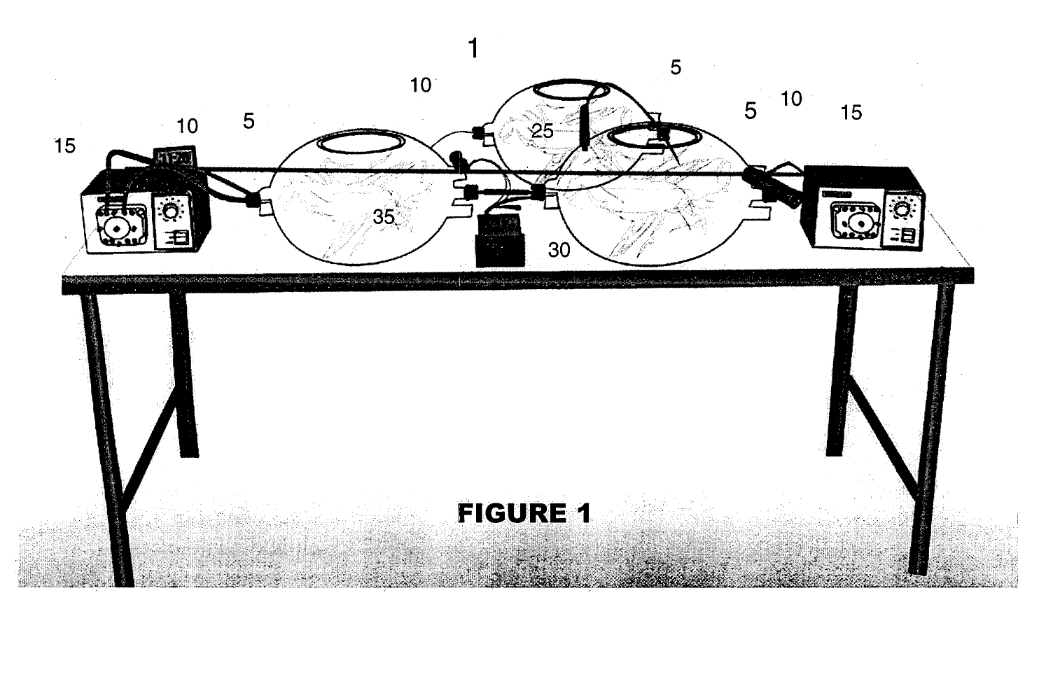

Also, provided is a specialized apparatus for expanding, growing, maintaining, sustaining and maturing cells. The specialized apparatus is termed a bioreactor chamber, containing at least one chamber, in particular, at least two chambers. The chambers of the bioreactor are connected by tubing that allows bi-directional flow of fluid between the chambers. The pressure driving the bi-directional flow of fluid is provided by at least by one peristaltic pump with a multiple head, or two or more stand-alone pumps. Other ancillary systems, not described herein in FIG. 1, are also used including a micro-oxygenator and pump, a CO₂ reserve and some type of molecular sieve filtration system. Alternatively, pumps are associated with each chamber for even flow distribution.

Since the invention provides MGCs generated by any animal, the invention provides methods of using the MGCs to contribute to therapeutics in vivo and in vitro comprising, injecting the primed MGCs into the host animal or mammal.

Preparations of the MGCs can be derived from the same species or they can be derived from different species. Translocation of the primed MGCs can be into the same species host or a different species host.

Alternatively, the primed MGCs can be used to derive cells for therapeutics to treat abnormal conditions and tissue repair.

### EXAMPLES

All the cell types and other materials not described herein are obtained through available sources and/or through standard methods used in the art.

Unless defined otherwise, all technical and scientific terms used herein have the same meaning as commonly understood by one of ordinary skill in the art to which the invention belongs.

All publications mentioned herein are incorporated herein by reference to describe and disclose specific information for which the reference was cited in connection with and are not to be construed as an admission that the invention is not entitled to antedate such disclosures by virtue of prior invention.

Throughout this description, the preferred embodiment and examples shown should be considered as exemplars, rather than as limitations on the present invention.

### Example 1

Isolating the Primordial Sex Cells (PSCs). The mammal or animal is anesthetized and the gonads are removed and transected. The primary sex cells (PSCs) are isolated with the aid of a microscope. Alternatively, a biopsy punch of the gonads can also be used and the PSCs isolated with the aid of a microscope. Under the microscope the PSCs have stem cell morphology (i.e. large, round and smooth) and are mechanically retrieved from the gonads. In particular, the spermatogonia and oogonia, are retrieved from the gonads. In particular, type A and type B spermatogonia are retrieved.

To obtain an ova/ovum, the animal is superovulated, and at least one ovum is retrieved and placed in nutritive media to keep it viable. The ova is held in place using a micropipette and with another micropipette (i.e. patchman) enter the ova until the tip is adjacent to the ova nucleus. Enucleating the ova is possible by applying a small vacuum to the micropipette. Discard the ova (1n) nucleus. Enucleation methods (above) are repeated with the PSCs (i.e spermatogonia and/or oogonia), except this time the nucleus is retained and the cytosol is discarded.

Other methods of enucleation and nucleation are contemplated within the scope of the present invention including other mechanical methods as well as methods utilizing electrical stimuli.

Creating the Modified Germ Cell (MGC). In a culture dish containing nutritive media the enucleated ovum is held in place using one micropipette and with another micropipette the nucleus from the PSC is inserted into the enucleated ovum. The PSC/ova is now termed a modified germ cell or MGC.

Enucleated or nucleated ova/ovum and PSCs can be stored by cryo-protection. The ova/ovum and PSCs can be thawed and used at a later time.

There are alternative methods to renucleate a cell including cell fusion methods, all which is within the scope the present invention.

### Example 2

Expanding the MGC. Take the MGC and place in a nutritive media comprising at least M15:high glucose DMEM (minus pyruvate and minus glutamine), about 15-20% fetal bovine serum (FBS), 1×1-glutamine, 1×penicillin/streptomycin, 1×non-essential amino acids, 1×ribonucleosides, 1×b-mercaptoethanol (bME) and 1:1000 leukemia inhibitory factor (LIF). The MGCs are prohibited from aggregating past a certain cell size, for example, the 6-cell stage. Mitotic divisions of the MGC can be observed using a dissecting scope. Although cell aggregates equal to or greater than 10² are possible, it is not preferred in the present invention. Also, the MGCs can be expanded on a layer of feeder cells. However, typically the cells are kept in suspension when maintained in the bioreactor chamber. At the 6-cell stage, the MGCs are mechanically separated. Alternatively, a sugar residue that binds to the surface molecules on the membrane of the MGCs will prevent aggregation of cells. Other methods of separating the MGCs are also within the scope of the present invention.

The MGCs at this 6-cell stage are now grown or expanded to lesser to equal 10³ to greater and equal to 10⁸ cells, preferably 10⁵ cells. The MGCs should be suspended in a multicellular or unicellular state in the bioreactor chamber(s). Upon expanding to certain number, the MGCs are then placed in a different bioreactor chamber containing a different medium to start the maturation process.

### Example 3

The Bioreactor Chamber. FIG. 1 describes an example of one modification of the bioreactor chamber. As mentioned above the bioreactor is comprised of at least one chamber, preferably at least two chambers. The chamber is used to grow, expand, maintain, sustain and mature cells, generally, or in the case of the present invention, MGCs.

FIG. 1 is an example of a multi-chambered bioreactor 1. The chambers 5 can be limited to one, but preferably there are at least two chambers 5. The chambers 5 are comprised of silicon oxide or glass. However, other materials used to construct similar biological chambers can be used. The chambers 5 are connected by tubing 10 to each other, and further connected by tubing 10 to various ancillary systems including peristaltic pumps 15, micro-oxygenators, CO₂ reserves and molecular sieve filters (not shown in FIG. 1). The tubing 10 is comprised of neoprene or other similar made materials for use in biological systems. The tubing 10 can have various diameters from ⅛ of an inch to ⅓ of an inch. However, smaller or greater diameter tubing for similar uses is possible. The different size tubings 10 are accommodated by different size fittings 20 of the chamber(s) 5. The tubing 10 allows flow of fluid media in the chambers comprising of nutrients, further comprising of macro and micromolecules, between the chambers 10. The flow of the nutrients is driven by two peristaltic pumps 15; or alternatively by at least one pump with multiple heads (not shown). Each peristaltic pump 15 or each head of a multi-head peristaltic pump drives fluid flow in one direction. However, using at least two pumps 15 allows for bi-directional fluid flow into and out of the chambers 5.

Also shown in FIG. 1 is a pH sensor 25 and pH meter 30. The pH sensor 25 is first connected to a pH meter 30 is secondly connected or immersed below the surface of the media in the chamber 5. Although not described in FIG. 1, a pH sensor 25 is optionally immersed below the surface of the media in all chambers 5 of the bioreactor 1 and further connected to a pH meter 30. The pH sensor 25 detects drops and rises in pH in the media in the chamber 5, and will send a stimulus to the pH meter 30. The pH meter 30 in turn contains wires connected to CO₂ valves 35 further connected by fittings 20 on the chambers 5. For example, when the pH of the media in the chambers 5 is low, a stimulus back to the pH meter 35 to open the CO₂ valve(s) 35, thereby allowing CO₂ from the CO₂ reserve to flow into the chamber 5.

Ancillary systems not shown in FIG. 1, include a CO₂ reserve which supplies CO₂ via the CO₂ valve 35. Also not shown in FIG. 1 is a micro-oxygenator (Aqua Pro) and pump. The micro-oxygenator is connected similar to the CO₂ reserve via a valve and tubing 10. Fluid from the tubing 10 flows through the micro-oxygenator and is oxygenated by side ports or inlets which inject oxygen into the space; thereby aerating the fluid for improved viability of the cells.

Also not shown in FIG. 1 is a molecular dialysis filter. Similar to the micro-oxygenator and attachment, fluid flows through the filter and particular sized molecules are restricted, for example, molecules at least about 60 KDa are restricted from the fluid. The dialysis filter works on counter-current system and unidirectional current system.

In addition, highly purified water (i.e., ionized, UV treated and microfiltered) is used to sustain the proper water content in the system: The-highly purified water can be added to the media in the chambers 5 by any sterile means available.

The media used in the chambers 5 is artificial blood comprised of a perfluorocarbon, which affords increased oxygen to the cells.

Fetal blood from different stages of development can be used as media and/or supplements to the media. In another aspect of the present invention, fetal blood is used to bathe the stem cells in vivo by placing MGCs in an anchored semi-permeable membrane.

In another aspect of the present invention, the proteins and macro- and micromolecules can be extracted using electrophoresis and filtration methods standard in the art. Additionally, the proteins and macro- and micro-molecules can also added to the fluid media in the chambers for the purposes of maturing in an acellular environment.

Using the Bioreactor Chamber 1 to Mature the MGCs. To mature the MGCs, various fetal cells from the earliest part of gestation on through all the different stages of tissue development are isolated from the animal. The animal, for example, can be the same species of mammal or different mammal. The fetal cells are maintained in one bioreactor chamber 5, which is nearby the chamber 5 which houses the MGCs. This chamber 5 contains embryonic stem (ES) cell media as previously described. In a nearby chamber 5, the expanded MGCs are maintained in a similar ES cell media. As shown in FIG. 1, there is tubing 10 communicating the chambers 5, thereby allowing free flow of nutrients from one chamber 5 into the compartment of the other chamber 5. For example, cells from the blastula are removed from the donor mammal or animal, and placed in a chamber 5 containing ES cell media. These blastula cells continue to divide and differentiate. During this developmental process, these blastula cells are secreting various macro and micromolecules (i.e. cytokines, GFs, different proteins) which then diffuse to the nearby chamber 5 wherein the MGCs are being sustained. This free flow of messengers facilitates the maturation of the MGCs, such that over a period of time, the MGCs develop various receptors or other surface markers to respond to the messengers secreted by the fetal cells in the nearby chamber 5.

This procedure can be repeated several times with different fetal cells from different stages. When replacing the fetal cells with different fetal cell types, the chambers 5 are closed off by means of a valve on the fittings 20. The ancillary systems including the CO₂ reserve, the micro-oxygenator and the molecular dialysis filter are shut off. The cells and media are removed from all chambers 5 in use, and new media and new fetal cells are replaced. The maturing MGCs from the previous process with the first fetal cell stage are placed back in a nearby chamber 5. The valves are re-opened and the ancillary systems and pumps turned back on. Fluid again fluid flows freely between the chambers 5 and driven by the peristaltic pumps 15.

### Example 4

Screening the MGCs. To screen for receptors on the MGCs, samples are taken from the chambers 5 containing the MGCs. Samples can be taken at different intervals for the purpose of determining the level of maturation MGCs. The level of maturation of the MGCs is determined by the number of receptor sites on the membrane of the MGCs and location of the receptor sites.

Fluorescence Resonance Energy Transfer (FRET) and Bioluminescence Resonance Energy Transfer (BRET) are technologies based on Resonance Energy Transfer (RET). It has been reported that energy transfer efficiency is highly dependent on the distance between the donor and acceptor moieties and their relative orientation with respect to each other. In most RET-based assays, the typical effective distance between the donor and acceptor is 10 to 100 angstroms and this range correlates with most biological interactions. (BRET; Packard BioScience, BioSignal Packard Inc., Meriden, Conn.). The use of BRET and FRET technologies, screen for MGCs with certain numbers of receptors and their location on the cell. Visual identification of receptors using BRET and FRET can be viewed on a larger screen or monitor. These projection systems are standard in the art.

Alternatively, other methods to screen for receptors sites in contemplated within the present invention, although not described herein.

Ultimately, the MGCs have developed all, or nearly all, or mostly all the receptor sites as that observed on a mature stem cell. Often the maturing and screening procedures are repeated many times depending on the number of types of fetal cells utilized and the length of growth or development in any one media. That is the number of steps is on a case by case basis.

### Example 5

Translocation into the Host. Once the MGCs have acquired the critical mass of receptors to develop into a mature stem cell (primed MGCs), they are subsequently translocated into the host tissue, in vivo or in vitro. For example, the primed MGCs are injected systemically into the circulatory system of the host in vivo. In the circulatory system, the primed MGCs typically migrate to the bone marrow where other adult stem cells reside. However, similar to other adult stem cells in the bone marrow, the primed MGCs travel systemically and will relocate where they are needed.

In another aspect of the present invention, the mature MGCs are injected subcranially into the CNS.

In one aspect of the present invention, the primed MGCs are translocated in the host of the same species as the donor which contributed the PSCs to create the MGC. This approach is preferred since there is no immune rejection from that of the recipient host against the donor primed MGCs.

### Example 6

Retrieving the primed MGCs. The MGCs are first placed in a semi-permeable membrane compartment. Affixed to the membrane compartment is a thin tagged thread. For example, the semi-permeable bag with the thread affixed is translocated into an animal in vivo or a tissue in vitro. The thread is tagged by fluorescence, so when exposed to UV light, the thread fluoresces and is visualized. The compartment is retrieved by locating the tagged thread. Subsequent screening of the primed MGCs is optionally performed to determine their level of development.

In another example, the membrane compartment or the thread can be comprised of a hollow fiber.

Alternatively, another bioassay to screen for the number of receptors on MGCs or primed MGCs is accomplished by placing the MGCs in post-natal tissue (i.e. skin) and determining the linearity of the precursor cells that have developed from the MGCs in the tissue type.

Accordingly, the invention is not limited to the precise embodiments described in detail hereinabove.

For example, allowing the MGCs to become 10⁵ before translocation into the host is detailed above, however, any number greater than or less than 10⁵ will also work under certain conditions and depending on the donor and host animal.

Also, many of the methods described herein are performed in vivo in an animal, in particular a mammal. However, methods in vitro are contemplated within the scope of the present invention.

Again, while the specification describes particular embodiments of the present invention, those of ordinary skill can devise variations of the present invention without departing from the inventive concept.

We claim:

1. A modified germ cell comprising:
a primordial sex cell (PSC), or nucleus thereof, translocated into an enucleated ovum, wherein the PSC and the ovum are derived from the same species or different species of animal.

2. The modified germ cell according to claim 1 wherein the PSC is a spermatogonium.

3. The modified germ cell according to claim 1 wherein the PSC is an oogonium.

4. The modified germ cell according to claim 1 wherein the animal is selected from the group consisting of rodents, primates, felines, canines, equines, porcine, bovines, and ovine.

5. The modified germ cell according to claim 1 wherein the cell is totipotent.

6. The modified germ cell according to claim 1 wherein the wherein the cell is pluripotent.

7. The modified germ cell according to claim 1 wherein the wherein the cell is multipotent.

8. The modified germ cell according to claim 1 wherein the wherein the cell is bipotent.

9. A modified germ cell comprising:
a spermatogonium, or nucleus thereof, translocated into an enucleated ovum, wherein the spermatogonium and the ovum are derived from the same species of animal.

10. A modified germ cell comprising:
a oogonium, or nucleus thereof, translocated into an enucleated ovum, wherein the oogonium and the ovum are derived from the same species of animal.

11. A method for preparing a modified germ cell comprising:
(a) obtaining a PSC from a first donor animal;
(b) obtaining an ovum from a second donor animal of the same species as the first donor animal;
(c) enucleating the ovum; and
(d) translocating the PSC, or nucleus thereof, into the enucleated ovum.

12. The method for preparing a modified germ cell according to claim 11 wherein the PSC obtained from the first donor animal is a spermatogonium.

13. The method for preparing a modified germ cell according to claim 11 wherein the PSC obtained from the first donor animal is an oogonium.

14. The method for preparing a modified germ cell according to claim 11, further comprising cryopreserving the modified germ cell.

15. A method for maturing a modified germ cell comprising:
(a) expanding the modified germ cell in a first medium;
(b) placing the modified germ cell into a second medium;
(c) screening for membrane receptors on the modified germ cell;
(d) repeating steps b and c until the number of receptors on the modified germ cell are sufficient to be a primed modified germ cell; and
(e) translocating the primed modified germ cell into the tissue of the animal;

16. The method for maturing the modified germ cell according to claim 16, wherein the second medium is derived from a stage one maturing cell(s) from an animal.

17. The method for maturing the modified germ cell according to claim 15, wherein the second medium is derived from a stage two maturing cell(s) from an animal.

19. The method for maturing the modified germ cell according to claim 15, wherein a second media type is dependent on the stage of maturing cell(s) from an animal.

20. The method for maturing the modified germ cell according to claim 15, wherein the animal is a mammal of the same species as the first donor mammal.

21. The method for maturing the modified germ cell according to claim 15, wherein the animal is a mammal of a different species as the first donor mammal.

22. The method for maturing a modified germ cell comprising:
(a) obtaining a PSC from a first donor mammal;
(b) obtaining an ovum from a second donor animal of the same species as the first donor animal;
(c) enucleating the ovum;
(d) translocating the PSC, or nucleus thereof, into the enucleated ovum;
(e) expanding the modified germ cell in a first medium;
(f) placing the modified germ cell into a second medium;
(g) screening for membrane receptors on the modified germ cells;
(h) repeating steps b and c until the number of receptors on the modified germ cell are sufficient to be a primed modified germ cell; and
(i) translocating the primed modified germ cell into the tissue of the animal.

23. The method for maturing the modified germ cell according to claim 22, wherein the first medium is derived from a stage one maturing cell(s) from an animal of the same species as the first donor animal.

24. The method for maturing the mammalian modified germ cell according to claim 22, wherein the second medium is derived from a stage two maturing cell(s) from an animal of the same species as the first donor animal.

25. The method for maturing the modified germ cell according to claim 22, wherein consecutive media is used from consecutively maturing cell(s) from an animal of the same species as the first donor animal until the modified germ cell has comparable receptors as that of the maturing cell.

26. A method for preparing a modified germ cell comprising:
(a) obtaining a PSC from a first donor mammal;
(b) obtaining an ovum from a second donor mammal of the same species of the first donor mammal;
(c) enucleating the ovum;
(d) fusing the PSC and the enucleated mammalian ovum to form a fused cell; and
(e) activating the fused cell.

27. The method for preparing a modified germ cell according to claim 26, wherein the activating step (e) comprises increasing intracellular levels of divalent cations and reducing phosphorylation of cellular proteins in the modified germ cell.

28. The method for preparing a modified germ cell according to claim 26, wherein the medium comprises antimycotic.

29. The method for preparing a modified germ cell according to claim 26, wherein the medium comprises antibiotic.

30. The method for preparing the modified germ cell according claim 26 comprises using an electrical stimulus.

31. A method for inducing differentiation of a modified germ cell comprising:
(a) obtaining at least one mammalian modified germ cell;
(b) culturing the modified germ cells in the presence of differentiating factors under suitable conditions, and for a time sufficient, to induce the cells to differentiate.

32. The method for inducing differentiation of a modified germ cell according to claim 31 wherein the modified germ cells are obtained by preparing them according to any one of claims 10 through 30.

33. The method for inducing differentiation of a modified germ cell according to claim 31 wherein the culturing step is performed in vitro.

34. The method for inducing differentiation of a modified germ cell according to claim 31 wherein the in vitro culture is conducted in a multi-chambered cell culture vessel such that the modified germ cells are isolated and separated from the source of the differentiating factors.

35. The method for inducing differentiation of a modified germ cell stem cell according to claim 31 wherein the source of differentiating factors are derived from cells and tissues.

36. The method for inducing differentiation of the modified germ cell according to claim 31 wherein the culturing step is performed in vivo.

37. The method for inducing differentiation of the modified germ cell according to claim 31 wherein the modified germ cells are induced to differentiate by implanting the modified germ cell into an animal.

38. The method for inducing differentiation of the modified germ cell according to claim 31 wherein the implanted modified germ cells are contained within a semi-permeable cell membrane such that the implanted modified germ cell remain isolated from the animal.

39. The method for inducing differentiation of the modified germ cell according to claim 31 wherein the semi-permeable cell membrane is retrieved by attaching a hollow fiber or thread to the membrane.

40. The method for inducing differentiation of the modified germ cell according to claim 31 wherein the hollow fiber or thread is tagged.

41. The method for inducing differentiation of the modified germ cell according to claim 31 wherein the tag is fluorescent.

42. A cell culture chamber comprising:
(a) at least one isolation chamber;
(b) tubing connecting the chamber; and
(c) at least one pump.

43. A cell culture chamber according to claim 42, wherein there is at least two chambers.

44. A cell culture chamber according to claim 42, wherein the chamber contains sufficient fluid appropriate to grow, expand, maintain, sustain and mature cells in vitro.

45. A cell culture chamber according to claim 42, wherein the fluid is further comprised of fetal blood.

46. A cell culture chamber according to claim 42, wherein the chambers are further comprised of different size fittings.

47. A cell culture chamber according to claim 42, wherein the chambers are connected by tubing via the fittings.

48. A cell culture chamber according to claim 42, wherein the fluid in the chamber flows between the chambers via the tubing and driven by the pumps.

49. A cell culture chamber according to claim 42, wherein the chamber is connected to a carbon dioxide source.

50. A cell culture chamber according to claim 42, wherein the chamber is connected to an oxygen source.

51. A cell culture chamber according to claim 42, wherein the chamber is further comprised of different size fittings.

52. A cell culture chamber according to claim 42, wherein there is a pH sensor detecting the hydrogen content of the fluid in the chamber.

53. A cell culture chamber according to claim 42, wherein the pH sensor is further connected to a pH meter that controls the amount of substance to be injected into the fluid in the chamber.

54. A cell culture chamber according to claim 42, wherein the substance is carbon dioxide.

55. A cell culture chamber according to claim 42, wherein the substance is any other substance which returns the pH of the fluid in the chamber to its desired pH.

56. A cell culture chamber comprising:
(a) at least one isolation chamber;
(b) tubing connecting the chamber;
(c) a carbon dioxide source;
(d) an oxygen source;
(e) a molecular filter; and
(f) at least one pump.

57. A cell culture chamber according to claim 56, wherein there is at least two chambers.

58. A cell culture chamber according to claim 56, wherein the chamber contains sufficient fluid volume appropriate to grow, expand, maintain, sustain and mature cells in vitro.

59. A cell culture chamber according to claim 56, wherein the chambers are further comprised of different size fittings.

60. A cell culture chamber according to claim 56, wherein the chambers are connected by tubing via the fittings.

61. A cell culture chamber according to claim 56, wherein the fluid in the chamber flows between the chambers via the tubing and driven by the pumps.

62. A cell culture chamber according to claim 56, wherein the chamber is further comprised of different size fittings.

63. A cell culture chamber according to claim 56, wherein there is a pH sensor detecting the hydrogen content of the fluid in the chamber.

64. A cell culture chamber according to claim 56, wherein the pH sensor is further connected to a pH meter that controls the amount of substance to be injected into the fluid in the chamber.

65. A cell culture chamber according to claim 56, wherein the substance is carbon dioxide from the carbon dioxide source.

66. A cell culture chamber according to claim 56, wherein the substance is any other substance which returns the pH of the fluid in the chamber to its desired pH.
